(19) United States
(12) Patent Application Publication
(10) Pub. No.: US 2020/0360260 A1
(43) Pub. Date: Nov. 19, 2020

(54) TOPICAL COMPOSITION COMPRISING ANTIMICROBIAL LIPID

(71) Applicant: Conopco, Inc., d/b/a UNILEVER, Trumbull, CT (US)

(72) Inventors: Chung-Ching CHU, Shanghai (CN); Mingming PU, Shanghai (CN); Yining XU, Shanghai (CN)

(73) Assignee: Conopco, Inc., d/b/a UNILEVER, Trumbull, CT (US)

(21) Appl. No.: 16/765,885

(22) PCT Filed: Nov. 15, 2018
(86) PCT No.: PCT/EP2018/081350

§ 371 Date: May 21, 2020

(30) Foreign Application Priority Data
Dec. 15, 2017 (CN) PCT/CN2017/116391
Jan. 25, 2018 (EP) 18153401.7

(51) Int. Cl. A61K 8/68; A61K 8/36; A61K 8/34; A61K 8/9789; A61Q 19/00

(52) U.S. Cl. CPC A61K 8/68; A61K 8/361; A61K 2800/10; A61K 8/9789; A61Q 19/00; A61K 8/347

(57) ABSTRACT

Disclosed is a topical composition comprising an antimicrobial lipid found in the sebum or stratum corneum of human beings, other than saturated C_{8 to 18} fatty acids, wherein said composition further comprises a biphenol obtainable from Magnolia spp. Also disclosed is a method of providing topical antimicrobial benefit comprising a step of applying a safe and effective amount of the topical antimicrobial composition

### FIELD OF THE INVENTION

This invention relates to topical antimicrobial compositions, especially cosmetic compositions and more particularly to cosmetic compositions having antimicrobial effect and useful at least against some microbes associated with conditions of cosmetic relevance such as dandruff and acne.

### BACKGROUND OF THE INVENTION

Human skin contains two important layers, a thicker layer called dermis and an outermost thinner layer called the epidermis. The dermis is responsible for strength, elasticity and thickness. With age, the thickness of dermis decreases, and this is believed to, at least in part, cause wrinkles. The epidermis contains a variety of cells which provide resilience and barrier properties. Keratinocytes account for 75 to 80% of cells in the epidermis.

The epidermis mainly contains three types of cells. They are the keratinocytes, the melanocytes and the Langerhans cells which are responsible for innate immunity.

The dermis provides support and is mainly made up of fibroblasts and extracellular matrix. Leucocytes, mast cells and tissue macrophages are also found therein. Finally, blood vessels and nerve fibers traverse the dermis.

Our skin acts as the primary line of defense against invading pathogens, like viruses, fungi and bacteria. As a primary organ of defense, the skin tissues always remain in contact with the environment and constantly face and resolve threats and challenges from invading pathogens. Therefore, the exposed surface is not only challenged by pathogenic foreign bacteria, but it also remains in contact and interacts with the resident commensal bacteria. Despite all challenges from the foreign and the commensal microbes, a healthy skin remains infection-free and the number of the resident microflora remains stable. This equilibrium in the interaction between the skin tissue and the microbes is maintained as the skin has sophisticated barrier property and defense strategy and skin lipids with anti-microbial properties (the antimicrobial lipids, AMLs) form an important part of that.

AMLs form an integral part of the skin's own defense system. AMLs exhibit a broad spectrum of activity against at least some of the bacteria, fungi, enveloped viruses and parasites.

It is known that skin lipids, more specifically, free fatty acids and long-chain sphingoid bases possess anti-microbial effect against Gram-Positive and Gram-Negative pathogenic bacteria (J Invest Dermatol. 98:269-273, 1992). Further, in vitro tests have shown that palmitoleic and lauric acids act against common skin pathogens, such as Pneumococci, Streptococcus, Corynebacteria, Micrococci, Candida and Staphylococcus aureus (Kabara et al., 1972, Antimicro. Agents and Chemo., 2, 23-28 and 1978, J. Soc. Cosmet. Chem., 29, 733-741).

However, due to changes in physiological, environmental, or pathological conditions there may be situations in which the barrier no longer remains adequate. Under such conditions the skin adapts and tries to augment the synthesis of its lipids. However, that may still not provide adequate protection. Therefore, some intervention might become necessary.

Dandruff and acne are globally prevalent problems. Dandruff is manifested by the shedding of clumps of dead skin cells from the scalp. These are white and readily visible therefore they present an aesthetically displeasing appearance. A factor that contributes to dandruff is certain members of the Malassezia yeasts. To combat them, various anti-dandruff compositions such as shampoos are available. Usually such shampoos contain surfactants and one or more anti-dandruff agent. Typical anti-dandruff agents are metal pyrithione e.g., zinc pyrithione (ZPTO), octopirox (piroctone olamine), azole antimicrobials (e.g. climbazole), selenium sulfide and combinations thereof.

While the problem of dandruff is mitigated to a large extent through use of the above actives in such shampoos, there is a need for more efficacious compositions.

Acne, also known as Acne vulgaris, is a common skin condition that affects nearly all adolescents and adults. It has a complex etiology involving abnormal keratinization and excess sebum production. Acne usually occurs in areas rich in sebaceous glands like the face, neck and back.

A bacterium named Propionibacterium acnes (P. acnes) is known as an important microbial agent associated with acne. Acne has been treated in many ways. Most treatments take several weeks to months before a noticeable change is seen. Benzoyl peroxide which has an antibacterial effect has been used for mild cases of acne and is also believed to prevent formation of further acne. In very severe cases of acne, antibiotics like tetracycline, erythromycin and clindamycin have been used.

US 2015/0373970 A1 (3M) discloses antimicrobial wipes containing, among other ingredients, an antimicrobial lipid such as a fatty acid ester, fatty ether, or alkoxide derivative thereof and an enhancer which the enhancer (preferably a synergist) functions to enhance the antimicrobial activity especially against Gram-Negative bacteria, such as E. coli and Pseudomonas sp. The enhancer affects the cell envelope of the bacteria and allows the antimicrobial lipid to more easily enter the cell cytoplasm and/or by facilitating disruption of the cell envelope. The enhancer is a soluble organic acid or its salt.

WO2014/131191 A1 (Johnson & Johnson) discloses cosmetic compositions having honokiol and/or magnolol along with carboxylic acids. The acids are included to enhance the deposition of the actives on the skin. The deposition is at least 100% more than compositions without the carboxylic acid and as high as 200% to 300% in certain embodiments.

US2013/0129643 A (Colgate) discloses an oral care composition for treating or preventing calculus comprising an anticalculus agent and an antibacterial agent comprising a biphenol compound obtainable from Magnolia officinalis.

CN104225603 (Tianjin Bokni Technology Dev Co Ltd) discloses paeonol and glyceryl polyether complex compound which is bactericidal.

KR 2015-0106804 (LG Household and Health Care) discloses personal cleansing composition comprising extract of magnolia and a fatty acid. In addition, the compositions also comprise a perfume having clogP value of at least 3, example nutmeg extract. The natural extract improves skin absorption and provides reverberation even after cleansing. The problem to be solved is to provide a composition for human body cleansing which exhibits strong antibacterial effect on body odor and acne bacteria including onium skin bacterium. the fatty acid is selected from the group consisting of C8-20 carboxylic acids including lauric acid, myristic acid, palmitic acid, stearic acid, oleic acid, linoleic acid and the like derived from animal or vegetable oils; fatty acids derived from vegetable oils including palm oil, palm kernel oil, soybean oil, rape oil, corn oil, rapeseed oil, sunflower oil, safflower oil, cottonseed oil, sesame oil, rice bran oil, camellia oil, castor oil, olive oil and palm oil; fatty acids derived from animal fats including tallow, lard, sunfish, fish oil, whale oil, and tuna oil, and the like (for example, tallow acid); fatty alcohols, mineral oils and paraffin.

JP2014172848 A2 (Kao Corp) discloses a bactericidal agent which has persistent bactericidal effect against Staphylococcus aureus. It comprises a combination of calcium palmitoleate and calcium linoleate as active ingredients.

WO9849999 A2 (Cosmoferm BV) discloses inhibition of growth of topically occurring microbes by application of a topical composition comprising a sphingoid base formulated in combination with a surfactant.

### SUMMARY OF THE INVENTION

We have determined that when certain antimicrobial lipids which are usually found in the sebum or stratum corneum of human beings are formulated together with a biphenol obtainable from Magnolia spp, the combination is highly efficacious against some microbes associated with conditions like acne or dandruff. That led us to the inference that compositions comprising the abovementioned combination could have antidandruff, antiacne and general antimicrobial activity and could be suitable for use in e.g., hand sanitisers. The antimicrobial lipids referred to hereinabove are other than those which are the saturated C8-C18 fatty acids, for example lauric acid.

Therefore, in accordance with a first aspect is disclosed a topical composition comprising an antimicrobial lipid found in the sebum or stratum corneum of human beings, other than saturated C_{8 to 18} fatty acids, wherein said composition further comprises a biphenol obtainable from Magnolia spp.

In accordance with a second aspect is disclosed use of a composition of the first aspect as a topical antimicrobial composition.

In accordance with a third aspect is disclosed a method of providing topical antimicrobial benefit comprising a step of applying a safe and effective amount of a topical antimicrobial composition of the first aspect.

### DETAILED DESCRIPTION OF THE INVENTION

The compositions according to the present invention comprise a topically-acceptable carrier, vehicle or diluent which can have a variety of different forms. The topically-acceptable carrier should preferably be non-irritant. “Topically-acceptable” therefore means that the carrier is suitable for topical application to the skin without causing any untoward safety or toxicity concerns. In other words, these carriers are suitable for use on mammalian skin. The typical carrier can be in the form of a hydro-alcoholic system (e.g. liquids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light cream and heavy creams. The emulsions can also include microemulsion systems. Other suitable topical carriers include anhydrous solids and semisolids (such as gels and sticks); and aqueous based mousse systems. Nonlimiting examples of the topical carrier systems useful in the present invention are described hereinafter.

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of or “composed of.” In other words, the listed steps or options need not be exhaustive. It is noted that the examples given in the description below are intended to clarify the invention and are not intended to limit the invention to those examples per se. Similarly, all percentages and ratios contained herein are weight/weight percentages unless otherwise indicated.

Except in the operating and comparative examples, or where otherwise explicitly indicated, all numbers in this description indicating amounts of material or conditions of reaction, physical properties of materials and/or use are to be understood as modified by the word “about”. Unless specified otherwise, numerical ranges expressed in the format “from x to y” are understood to include x and y. When for a specific feature multiple preferred ranges are described in the format “from x to y”, it is understood that all ranges combining the different endpoints are also contemplated. The various features of the present invention referred to in individual sections above apply, as appropriate, to other sections mutatis mutandis. Consequently, features specified in one section may be combined with features specified in other sections as appropriate. Any section headings are added for convenience only, and are not intended to limit the disclosure in any way.

By a topical composition is meant a composition for external application in the form of a leave-on or wash-off format meant for cleaning or disinfecting topical areas e.g. skin and/or hair and or oral cavity of mammals, especially humans. Such a composition includes any product applied to a human body for also improving appearance, cleansing, odor control or general aesthetics. In accordance with one aspect, the compositions in accordance with the invention are rinse off compositions. Alternatively, they are leave-on compositions. The compositions of the present invention could be in the form of a liquid, lotion, cream, foam or gel, or toner, or applied with an implement or via a face mask, pad or patch. “Skin” as used herein is meant to include skin on the face and body (e.g., neck, chest, back, arms, underarms, hands, legs, buttocks and scalp). The composition of the invention is also of relevance to applications on any other external substrates of the human body other than skin e.g. hair.

By ‘an antimicrobial composition’ as used herein, is meant to include a composition for topical application to skin, hair and/or scalp of mammals, especially humans. Such a composition is generally applied on to the desired topical surface of the body for few seconds to up to 24 hours. When the time of application is low say of the order of a few seconds to a few minutes after which the composition is rinsed off with water or wiped away, such a composition is known as a cleansing composition or a rinse-off composition. When the composition is applied for longer time say from several minutes to up to 24 hours and washed off usually during the process of normal personal cleaning, such a composition is known as a leave-on composition. It is more preferably used for preventing or alleviating the symptoms of dandruff on the scalp and/or hair, for antiacne benefit, or for disinfecting the hand or other parts of the human body.

“Hair Care Composition” as used herein, is meant to Include a composition tor topical application to hair. Non-limiting examples of such compositions include leave-on hair lotions, creams, arid wash-off shampoos, conditioners, shower gels, or a toilet bar. When the composition of the invention is a hair care composition, it preferably is a wash-off composition, especially shampoo or a conditioner.

The Topical Compositions of the Invention

The invention relates to a topical composition which comprises an antimicrobial lipid found in the sebum or stratum corneum of human beings, other than saturated C_{8 to 18} fatty acids.

Antimicrobial lipids (AMLs) are present naturally in sebum and stratum corneum of human beings. They are secreted by the sebaceous glands on the skin or scalp or in the oral cavity. AMLs are also derived from keratinocytes and carried into the stratum corneum at the skin's surface. Most of the lipids of the skin are derived from the sebaceous glands which excrete an oily and waxy material called sebum. Most of the remaining lipids come from the stratum corneum cells of the epidermis. The contribution of lipids from each of these sources depends upon the number of sebaceous glands present at a given location. Reports indicate that there could be as many as 900 glands/cm² on the scalp or face and about 50 glands/cm² for the forearm.

Published literature indicates that the antimicrobial lipids present in the sebum or stratum corneum of the skin are tri-, di- and mono-glycerides, un-esterified fatty acids which may be saturated or unsaturated, long chain sphingoid bases, glyco- and phospholipids. The antimicrobial lipids act as a first line of defense against pathogenic microbes.

It is common to find published literature or commercially and publicly available topical compositions that contain one or more of such antimicrobial lipids. However, very little is known about attempts to boost or increase the innate activity of the antimicrobial lipids present in the sebum or stratum corneum.

The Minimum Inhibitory Concentration (MIC) is the classical approach to study and assess antimicrobial efficacy of various compounds. In microbiology, minimum inhibitory concentration (MIC) is the lowest concentration of an antimicrobial (like an antifungal, antibiotic or bacteriostatic) agent that will inhibit the visible growth of a microorganism after desired incubation time.

The concept of MIC applies also to antimicrobial lipids found in the sebum or stratum corneum of human beings. This may be considered as an indicator of the innate antimicrobial activity of such a lipid. Formulations containing antimicrobial lipids have been disclosed in past, but the present inventors are not informed about any publication or a publicly available product which contains one or more antimicrobial lipids and another ingredient(s) to reduce/lower the MIC of the concerned antimicrobial lipid. The invention therefore provides technically superior compositions in which the formulation scientists are at liberty to reduce the dosage of the antimicrobial lipids whilst still being able to claim effective protection against microbes.

It is believed that while the antimicrobial lipids present in the compositions of the invention replenish or augment their innate or naturally present counterparts in the sebum or stratum corneum, the biphenol obtainable from Magnolia spp interacts synergistically with the lipids to make them even more efficacious.

While the concerned antimicrobial lipids could be any lipid that is found on or in the sebum or stratum corneum, it is preferred that the lipid is at least one of sapienic acid, sphingosine, dihydrosphingosine, phytosphingosine, 6-hydroxy phytosphingosine or palmitoleic acid. More preferably the compositions of the invention comprise a lipid which is palmitoleic acid, sapienic acid or phytosphingosine.

Sapienic acid and palmitoleic acid are generally added into the antimicrobial agents in purified form. These fatty acids may be chemically synthesized or naturally sourced and can be in free fatty acid form or in their esterified form, but it is preferred that the lipids are in their acid form. Natural substances which contain these fatty acids and its derivatives may come from animal or plant sources, including but not limited to plant seed extracts, marine oils, animal oil and microbial ferments. Specifically, sapienic acid is reported as a major constituent of the seed oil of Thunbergia alata (85% by weight as the triglyceride). Therefore, sapienic acid may be added in the form of a seed oil extract comprising sapienic acid, while ensuring that sapienic acid is present in the desired concentration in the compositions of the present invention.

Sphingosine, phytosphingosine, and dihydrosphingosine, are sphingoid bases present in the human skin. They can be synthesized chemically or produced via suitable biotechnological process that lead to formation of sphingoid bases by yeast from materials like sugar. Sphingoid bases may be added to the antimicrobial composition in purified form. Alternatively, fermentation broth consisting of high quantities of sphingoid bases may be used, while ensuring that sphingoid bases are present in the desired concentration in the compositions of the present invention.

It is preferred that the amount of the lipid in the compositions of the invention is from 0.01 to 10 wt %. More preferably the amount is 0.01 to 5% by weight of the composition and most preferably 0.1 to 2%. When two or more antimicrobial lipids are present in the compositions of the invention, their total amount is as disclosed above.

Biphenol from Magnolia spp.

The compositions in accordance with the invention further comprise a biphenol obtainable from Magnolia spp. It is preferred that the amount of said biphenol obtainable from Magnolia spp is 0.01 to 10 wt %. The expression ‘a biphenol’ is used to indicate the presence of one of more compounds belonging to a generalized class of compounds having the basic structural formula of biphenol.

As the compositions comprise an antimicrobial lipid and a biphenol, it is preferred that a ratio is maintained between the amount of the active ingredients to get optimal efficacy. Preferably the ratio of the amount of said lipid to said biphenol is 0.003 to 125.

Preferably the compositions of the invention comprise water- or hydro-alcoholic extract of the bark of Magnolia, which in turn comprises said biphenol. A measured amount of the extract is chosen so that the requisite amount of the biphenol contained therein get included in the compositions.

It is preferred that the biphenol is at least one of magnolol, honokiol, tetrahydromagnolol, tetrahydrohonokiol, propylmagnolol, propylhonokiol, isopropylmagnolol, isopropylhonokiol, butylmagnolol, and butylhonokiol. It is particularly preferred that the biphenol is at least one of honokiol or magnolol. Alternatively, and further preferably the composition comprises honokiol and magnolol. It is preferred that the composition comprises 0.01 to 10 wt % of said honokiol or 0.01 to 10 wt % of said magnolol, provided that when said composition comprises both honokiol and magnolol, then their combined amount is 0.01 to 10 wt % of said composition.

In general, Magnolia is a large genus of about 210 flowering plant species in the subfamily Magnoliodieae of the family Magnoliaceae. Magnolia extract can be obtained from the species within the Magnoliaceae family. Non-limiting examples of these species include Magnolia acuminata, Magnolia ashei, Magnolia biondii, Magnolia cylindrica, Magnolia cambellii, Magnolia denudata, Magnoliapaseri, Magnolia grandiflora, Magnolia hypoleuca, Magnolia kobus, Magnolia hliiflora, Magnolia loegneri, Magnolia macrophylla, Magnolia officinalis, Magnolia pyramidata, Magnolia sargentiana, Magnolia seiboldii, Magnolia soulangiana, Magnolia sprengeri, Magnolia stellata, Magnolia tripetala, Magnolia virginiana, Magnolia zenii, and Michelia figo.

Magnolia extracts containing magnolol and/or honokiol are commercially available from a variety of different sources. For example, an extract of the bark of Magnolia officinalis which comprises both honokiol and magnolol, each in a concentration of about 45% (total concentration of honokiol and magnolol combined in the extract is 90%), is available from HNSEA under the tradename MagnoPro®. Individual magnolol and honokiol compounds isolated from the magnolia bark are available from H-NSEA under the tradenames “Magnolol 95% and “Honokiol 95%”, respectively. Alternatively, a person of ordinary skill in the art would be able to isolate Magnolia extract from the Magnolia flower, bark, or seed cone by using any suitable isolation and purification method known in the art. In some embodiments, the extract of magnolia may be obtained from dried Magnolia plant bark and can be prepared by any means known or to be developed in the art.

The sum of the fractional inhibitory concentrations (ΣFIC) is widely used in the context of combinations of antimicrobial ingredients. It is a tool to determine whether the antimicrobial ingredients (when used in combination) have synergistic effect or antagonistic effect or neither of the two, i.e., an additive effect. The present inventors have resorted to the ΣFIC test to evaluate the combined effect of honokiol and magnolol (both biphenols) with five different AMLs (sapienic acid, palmitoleic acid, sphingosine, phytosphingosine and dihydrosphingosine) against M. furfur, S. aureus and P. acnes.

It is preferred that the topical composition of the invention comprises sapienic acid and honokiol. In this case it is preferred that weight ratio of the amount of honokiol to that of sapienic acid is 0.25 to 1, alternatively 0.008 to 2.

Alternatively, the composition comprises sapienic acid and magnolol. In this case it is preferred that the weight ratio of the amount of magnolol to that of sapienic acid is 0.06 to 2, alternatively 0.01 to 4 and further alternatively 0.25 to 1.

Alternatively, the composition comprises sphingosine and honokiol. In this case it is preferred that the weight ratio of the amount of honokiol to that of sphingosine is 1.25 to 40, alternatively 0.8 to 25 alternatively 0.7 to 13.

Alternatively, the composition comprises sphingosine and magnolol. In this case it is preferred that the weight ratio of the amount of honokiol to that of sphingosine is 5 to 40, alternatively 1.5 to 25, alternatively 1.5 to 13.

Alternatively, it is preferred that the topical composition of the invention comprises dihydrosphingosine and honokiol. In this case it is preferred that weight ratio of the amount of honokiol to that of dihydrosphingosine is 5 to 40, alternatively 0.8 to 12.5 and alternatively 0.2 to 6.5.

Alternatively, the composition comprises dihydrosphingosine and magnolol. In this case it is preferred that the weight ratio of the amount of honokiol to that of dihydrosphingosine is 5 to 80, alternatively 0.4 to 12.5 and further alternatively 0.7 to 6.5.

Alternatively, it is preferred that the topical composition of the invention comprises phytosphingosine and honokiol. In this case it is preferred that weight ratio of the amount of honokiol to that of phytosphingosine is 10 to 80, alternatively 0.4 to 12.5 and alternatively 0.2 to 3.2.

Alternatively, the composition comprises phytosphingosine and magnolol. In this case it is preferred that the weight ratio of the amount of honokiol to that of phytosphingosine is 10 to 320, alternatively 0.2 to 12.5 and further alternatively 0.4 to 12.5.

Alternatively, it is preferred that the topical composition of the invention comprises palmitoleic acid and honokiol. In this case it is preferred that weight ratio of the amount of honokiol to that of palmitoleic acid is 0.06 to 2, alternatively 0.008 to 2 and alternatively 0.06 to 1.

Alternatively, the composition comprises palmitoleic acid and magnolol. In this case it is preferred that the weight ratio of the amount of magnolol to that of palmitoleic acid is 0.03 to 2, alternatively 0.008 to 2 and further alternatively 0.1 to 4.

Other Ingredients

The composition of the invention preferably comprises a cosmetically acceptable vehicle. The cosmetically acceptable vehicle is such that the composition can be prepared, e.g., as a shampoo, conditioner, body wash, hand wash or face wash product, cream, lotion, gel, powder, ointment, hand sanitiser or a soap bar and the rest of the ingredients would vary accordingly.

In one aspect the topical compositions in accordance with the invention is a hair care composition. Preferably such a composition is a shampoo, a hair conditioner, a hair serum or a hair oil. Most preferably the topical composition is an antidandruff composition effective against at least some Malessezia spp.

When the composition of the invention is a shampoo, it preferably comprises other ingredients which are generally included in such compositions.

A shampoo preferably comprises 1 to 20 wt %, more preferably 2 to 16 wt %, furthermore preferably from 3 to 16 wt % anionic surfactants, e.g. an alkyl sulphate and/or ethoxylated alkyl sulfate surfactant. Preferred alkyl sulfates are C₈₋₁₈ alkyl sulfates, more preferably C₁₂₋₁₈ alkyl sulfates, preferably in the form of a salt with a solubilising cation such as sodium, potassium, ammonium or substituted ammonium.

Particulartly preferred alkyl ether sulfates are those having the formula: RO(CH₂CH₂O)ₙSO₃M; wherein R is an alkyl or alkenyl having from 8 to 18 (preferably 12 to 18) carbon atoms; n is a number having an average value of greater than at least 0.5, preferably between 1 and 3, more preferably between 2 and 3; and M is a solubilising cation such as sodium, potassium, ammonium or substituted ammonium. An example is sodium lauryl ether sulfate (SLES). SLES having an average degree of ethoxylation of from 0.5 to 3, preferably 1 to 3 is especially preferred.

Shampoo compositions according to the invention may comprise one or more further anionic cleansing surfactants which are cosmetically acceptable and suitable for topical application to the hair. Examples include the alkaryl sulphonates, alkyl succinates, alkyl sulphosuccinates, alkyl ether sulphosuccinates, N-alkyl sarcosinates, alkyl phosphates, alkyl ether phosphates, and alkyl ether carboxylic acids and salts thereof, especially their sodium, magnesium, ammonium and mono-, di- and triethanolamine salts. The alkyl and acyl groups generally contain from 8 to 18, preferably from 10 to 16 carbon atoms and may be unsaturated. The alkyl ether sulphosuccinates, alkyl ether phosphates and alkyl ether carboxylic acids and salts thereof may contain from 1 to 20 ethylene oxide or propylene oxide units per molecule.

Typically, suitable anionic surfactants include sodium oleyl succinate, ammonium lauryl sulphosuccinate, sodium lauryl ether sulphosuccinate, sodium dodecylbenzene sulphonate, triethanolamine dodecylbenzene sulphonate, lauryl ether carboxylic acid and sodium N-lauryl sarcosinate. Suitable preferred additional anionic cleansing surfactants are sodium lauryl ether sulphosuccinate(n)EO, (where n is from 1 to 3), lauryl ether carboxylic acid (n) EO (where n is from 10 to 20).

Mixtures of any of the foregoing anionic cleansing surfactants may also be suitable. The shampoo composition of the invention preferably additionally comprises 0.1 to 10 wt %, more preferably from 0.5 to 8 wt % of an amphoteric surfactant, preferably a betaine surfactant such as alkyl amidopropyl betaine surfactant, for example cocoamidopropyl betaine.

The pH of the composition is preferably equal to or higher than 4.0, more preferably in the range of 5.0 to 10.0.

It is preferred that the shampoo composition additionally comprises 0.1 to 3 wt %, more preferably 0.1 to 1.5 wt % of a zinc compound. The presence of zinc in the composition is believed to improve the antidandruff efficacy. Suitable zinc compounds are ZPTO, zinc oxide, zinc citrate, zinc malonate, zinc carbonate or a combination thereof.

Preferably the shampoo composition additionally comprises 0.01 to 2 wt %, more preferably 0.025 to 0.75 wt % conazole fungicide. Preferably the conazole fungicide is ketoconazole or climbazole or mixture thereof. The presence of a conazole fungicide is believed to improve the deposition of zinc pyrithione (ZPTO).

The shampoo composition further preferably comprises a suspending agent. Suitable suspending agents are polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol distearate are preferred long chain acyl derivatives, since these impart pearlescence to the composition. Polyacrylic acid is available commercially as Carbopol® 420, Carbopol® 488 or Carbopol® 493. Polymers of acrylic acid cross-linked with a polyfunctional agent may also be used; they are available commercially as Carbopol® 910, Carbopol® 934, Carbopol® 941 and Carbopol® 980. An example of a suitable copolymer of a carboxylic acid containing monomer and acrylic acid esters is Carbopol® 1342. All Carbopol (trademark) materials are available from Goodrich.

Suitable cross-linked polymers of acrylic acid and acrylate esters are Pemulen® TR1 and Pemulen® TR2. A suitable hetero polysaccharide gum is xanthan gum, for example that available as Kelzan.

Mixtures of any of the above suspending agents may be used. Preferred is a mixture of cross-linked polymer of acrylic acid and crystalline long chain acyl derivative.

Suspending agent, if included, will generally be present at 0.1 to 10 wt %, preferably from 0.5 to 6 wt %.

A composition of the invention may contain other ingredients for enhancing performance and/or consumer acceptability. Such ingredients include fragrance, dyes and pigments, pH adjusting agents, pearlisers or opacifiers, viscosity modifiers, preservatives, and natural hair nutrients such as botanicals, fruit extracts, sugar derivatives and amino acids.

It is preferred that the shampoo composition is aqueous based. It preferably comprises 70 to 95 wt % water.

Hair Conditioner

As an alternative, the topical composition of the invention is a hair conditioner.

When conditioning benefits are to be delivered through the composition of the invention the composition is called a hair conditioner. Typically, the most popular conditioning agents used in hair care compositions are water-insoluble oily materials such as mineral oils, naturally occurring oils such as triglycerides and silicone polymers.

Conditioning benefit is achieved by the oily material being deposited onto the hair resulting in the formation of a film, which makes the hair easier to comb when wet and more manageable when dry. An especially useful conditioning agent is a silicone, preferably a non-volatile silicone. Advantageously compositions herein may include one or more silicones. The silicones are conditioning agents found in dispersed or suspended particulate form. They are intended to deposit onto hair remaining behind after rinsing of the hair with water. Suitable silicone oils may include polyalkyl siloxanes, polyaryl siloxanes, polyalkylaryl siloxanes, polyether siloxane copolymers and mixtures thereof. Amino silicones are often formulated with shampoo compositions. Amino silicones are silicones containing at least one primary amine, secondary amine, tertiary amine or a quaternary ammonium group. High molecular weight silicone gums can also be utilized. Another useful type are the crosslinked silicone elastomers such as DimethiconeNinyl/Dimethicone Crosspolymers (e.g. Dow Corning 9040 and 9041).

It is preferred that hair conditioner composition of the invention comprises 0.1 to 10 wt %, more preferably from about 0.1 to about 8 wt % silicone. Alternatively, the hair conditioner is silicone-free, containing not more than 1 wt % silicone. It is preferred that pH of the composition is more than 4.0, more preferably 5.0 to 7.0.

Hair conditioner composition of the invention preferably may also comprise 0.5 to 10 wt % fatty alcohol. The combined use of fatty alcohols and cationic surfactants in conditioning compositions is believed to be especially advantageous, because this leads to the formation of a lamellar phase, in which the cationic surfactant is dispersed.

Representative fatty alcohols comprise from 8 to 22 carbon atoms, more preferably 16 to 22. Fatty alcohols are typically compounds containing straight chain alkyl groups.

Examples of suitable fatty alcohols include cetyl alcohol, stearyl alcohol and mixtures thereof. The use of these materials is also advantageous in that they contribute to the overall conditioning properties of compositions of the invention.

Rinse-Off or Leave-On Compositions for Skin

In another aspect the antimicrobial composition of the invention is an antiacne composition effective at least against P. acnes. Further alternatively, it is a rinse-off or a leave-on composition effective at least against S. aureus.

Such compositions may be used for skin care e.g. body, hand or face care or as a personal wash composition like a shower gel. Alternatively, it is a hand sanitizer composition.

It is preferred that the composition in accordance with the invention comprise 5 to 80 wt % surfactant, which is a soap or a non-soap surfactant or a combination thereof. The nature of surfactants depends on the application. Preferably the surfactant is a nonionic surfactant, such as C₈-C₂₂, preferably C₈-C₁₆ fatty alcohol ethoxylates, comprising between 1 and 8 ethylene oxide groups when the product is in the liquid form. Alternatively, the surfactant is an anionic non-soap surfactant selected from primary alkyl sulphates, secondary alkyl sulphonates, alkyl benzene sulphonates, or ethoxylated alkyl sulphates. The composition may further comprise an anionic surfactant, such as alkyl ether sulphate preferably those having between 1 and 3 ethylene oxide groups, either from natural or synthetic source and/or sulphonic acid. Especially preferred are sodium lauryl ether sulphates. Alkyl polyglucoside may also be present in the composition, preferably those having a carbon chain length between C₆ and C₁₆.

It is particularly preferred that the surfactant is soap, more preferably from 5 to 90 wt %, furthermore preferably 10 to 85 wt %. Soap is a suitable surfactant for personal wash applications. Preferably the soap comprises C₈-C₂₄ soap, more preferably C₁₀-C₂₀ soap and most preferably C₁₂-C₁₈ soap. The cation of the soap can be alkali metal, alkaline earth metal or ammonium. Preferably, the cation of the soap is sodium, potassium or ammonium. More preferably the cation is sodium.

A typical fatty acid blend consists of 5 to 30% coconut fatty acids and 70 to 95% fatty acids. Fatty acids derived from other suitable oils/fats such as groundnut, soybean, tallow, palm or palm kernel may also be used in other desired proportions.

Preferably the antiacne composition, the rinse-off and the leave-on composition include other known ingredients such as perfumes, pigments, preservatives, emollients, sunscreens, emulsifiers, gelling agents and thickening agents. Choice of these ingredients will largely depend on the format of the composition.

When water is a carrier, it is preferred that the composition comprises 10 to 90 wt % water.

Inorganic particulate materials may also be a suitable carrier. When inorganic particulate material is the carrier, the antimicrobial compositions of the invention is solid. Preferably the inorganic particulate material is talc. When the inorganic particulate material is talc, the antimicrobial composition is particularly useful as a talcum powder for application on face or body.

Further alternatively, a solvent other than water may serve as preferred carrier. Although any suitable and cosmetically acceptable solvent can be used, alcohols are preferred. Short chain alcohols, in particular ethanol and propanol are particularly preferred as a carrier for an antimicrobial wipe or an antimicrobial hand sanitizer composition. The composition of the present invention may be in the form or wipes meant for personal hygiene.

Use and Method in Accordance with the Invention

In accordance with a second aspect is disclosed the use of a composition of the first aspect as a topical antimicrobial composition. Preferably the topical composition is an antiacne composition effective at least against P. acnes. Alternatively, the topical composition is an antidandruff composition effective against at least some Malessezia spp. Further alternatively the topical composition is rinse-off or a leave-on composition effective at least against S. aureus. It is preferred that the use in accordance with the invention is for non-therapeutic purpose. Preferably the non-therapeutic purpose means for cosmetic purposes. Alternatively, the use of the compositions in accordance with the invention is for therapeutic purpose. Persons ordinarily skilled in the art understand the difference between therapeutic and non-therapeutic uses of the compositions.

In accordance with another aspect is disclosed a method of providing topical antimicrobial benefit comprising a step of applying a safe and effective amount of a topical antimicrobial composition of the first aspect. The term safe an effective amount is well known to persons skilled in the art and such amounts may vary depending on the product format, for example, the said amount in the case of a hand sanitizer composition could be 1 to 2 ml for each application, while the same amount could be 5 to 10 ml for each application in the case of shampoos. Preferably the topical composition is an antiacne composition effective at least against P. acnes.

Alternatively, the topical composition is an antidandruff composition effective against at least some Malessezia spp. Further alternatively the topical composition is rinse-off or a leave-on composition effective at least against S. aureus. It is preferred that the method in accordance with the invention is for non-therapeutic purpose. Preferably the non-therapeutic purpose means for cosmetic purposes. Alternatively, the method of the compositions in accordance with the invention is for therapeutic purpose. Persons ordinarily skilled in the art understand the difference between therapeutic and non-therapeutic uses of the compositions.

In accordance with another aspect is disclosed a topical composition for use as an antiacne composition effective at least against P. acnes. In accordance with yet another aspect is disclosed an antidandruff composition effective against at least some Malessezia spp. In accordance with yet another aspect is disclosed a rinse-off or a leave-on composition effective at least against S. aureus.

The invention will now be described in detail with the help of the following non-limiting examples.

### EXAMPLES

The antimicrobial efficacy of exemplary compositions according to the invention was determined as against three organisms; M. furfur, S. aureus and P. acnes

The concerned procedures will now be briefly explained.

Method: ΣFIC Assay Against M. Furfur, S. Aureus and P Acnes

Step 1: Microbe Culture and Preparation

M. furfur (CBS1878) was inoculated into Pityrosporum Broth (PB, solution A) and incubated in a shaking incubator at 32° C. for 2 days. The suspension containing the microbe was then diluted 10-times by PB and incubated again at 32° C. for another 2 days. The inoculum was further diluted by PB to get around 10⁴ cells /ml. S. aureus (ATCC 12600) was inoculated into Tryptone Soya Broth (TSB, Oxoid: CM0129) and incubated in a shaking incubator at 37° C. for 1 day. The suspension containing the microbe was then diluted 10-times with TSB and incubated again at 37° C. for another 1 day. The inoculum was further diluted by TSB to get around 10⁵ cells/ml.

Propionibacterium acnes (ATCC 6919) was inoculated from freezer into liquid Reinforced Clostridial Medium (RCM, HepoBio: HB0316) for 3 days (anaerobic conditions) growth at 37° C. The suspension containing the microbe was then diluted 10-times with RCM and incubated again under 37° C. for another 3 days. The inoculum was further diluted by RCM to get around 10⁴ cells/ml.

Preparation of Solution A:

Pityrosporum Broth (PB)

| 10 g | Bacteriological Peptone |
|---|---|
| 0.1 g | Yeast extract |
| 10 g | Ox-bile |
| 2.5 g | Taurocholic acid |
| 10 g | Glucose |
| 1 L | Deionised water |
| 0.5 ml | Tween60 |
| 1 ml | Glycerol |
| Adjusted pH to 6.2 | |
| After sterilization | |
| 0.5 ml | UHT milk |

Step 2. Preparation of Stock Solutions of the Concerned Ingredient to be Tested

Honokiol (Ex. Sigma) 50 mg/ml in DMSO (50,000 ppm); Magnolol (Ex. Sigma) 50 mg/ml in DMSO (50,000 ppm); Sapienic acid (Ex. Parchem, CAS# 17004-51-2) 100 mg/ml in 100% ethanol (100,000 ppm); D-Sphingosine (Ex. Sigma S7049) 5 mg/ml in 50% ethanol (5,000 ppm); D-erythro-Dihydrosphingosine (Ex. Sigma D3314) 5 mg/ml in 50% ethanol (5,000 ppm); Phytosphingosine (Ex. Evonik) 5 mg/m stock in 50% ethanol (5,000 ppm); Palmitoleic acid (Ex. Sigma 76169) 100 mg/ml in 100% ethanol (100,000 ppm).

All preparations were checked to ensure complete dissolution before being aliquoted and stored at −20° C. for use.

Step 3: ΣFIC Test

The ΣFIC test was conducted based on the principle previously described in Hall M J, Middleton R F, & Westmacott D (1983), The fractional inhibitory concentration (FIC) index as a measure of synergy. Journal of Antimicrobial Chemotherapy 11(5):427-433. The procedure is as follows:

Twenty (20) μl of compound A (honokiol or magnolol as applicable to the test) and 20 μl of compound B (a selection of different AMLs as applicable to the test) were mixed in corresponding wells of a 96-well plate. An amount of 160 μl of microbial suspension was dispensed in each well and the broth medium as taken as a blank control for comparison of results. The total reaction volume in each well was 200 μl.

Thereafter the 96-well plates were incubated in an incubator. In accordance with the test protocols that were followed, M. furfur was incubated aerobically at 32° C. for 1 day, S. aureus was incubated aerobically at 37° C. for 1 day and P. acnes was incubated anaerobically at 37° C. for 4 days. Thereafter 20 μl alamar blue (0.1%) was dispensed in each well and the procedure of incubation (as abovementioned) was repeated. Finally, the change in colour of the indicator was monitored to check for visible signs of microbial growth or inhibition of the growth. If the colour changed to red it indicated growth (of microbes) and blue indicated no growth or inhibition of growth.

After all the observations were recorded and tabulated, the Fractional Inhibition Concentration (FIC) was calculated. The combination effect of inhibitory antimicrobials is widely explored using the concept of the Fractional Concentration (FC) and Fractional Inhibitory Concentration (FIC). This parameter is defined as follows:

ΣFIC=FIC (component 1)+FIC (component 2)

Further, the inference that could be drawn from the value of ΣFIC is summarised in the table below.

| | | |
|---|---|---|
| ΣFIC = 1 | additive antimicrobial activity | not acceptable |
| ΣFIC > 1 | antagonistic antimicrobial activity | not acceptable |
| ΣFIC < 0.9 | synergistic antimicrobial activity | inventive |

The final concentration of each ingredient in ppm (parts per million) after (10-fold dilution) was as follows. Each of these concentrations was tried out to determine the FIC value for (either of) honokiol or magnolol in combination with one of the AMLs.

Pertaining to M. Furfur:

Honokiol—5000, 2500, 1250, 625, 312.5, 156.25, 78.13, 39.07, 19.53, 9.77, 4.88 and 0.

Magnolol—5000, 2500, 1250, 625, 312.5, 156.25, 78.13, 39.07, 19.53, 9.77, 4.88 and 0.

Sapienic acid—10000, 5000, 2500, 1250, 625, 312.5, 156.3 and 0

D-Sphingosine—500, 250, 125, 62.5, 31.25, 15.63, 7.81 and 0

D-erythro-Dihydrosphingosine—500, 250, 125, 62.5, 31.25, 15.63, 7.81 and 0

Phytosphingosine—250, 125, 62.5, 31.25, 15.63, 7.81, 3.91 and 0

Palmitoleic acid—10000, 5000, 2500, 1250, 625, 312.5, 156.3 and 0

Pertaining to S. Aureus:

Honokiol—500, 250, 125, 62.5, 31.25, 15.63, 7.81, 3.91, 1.93, 0.98, 0.49 and 0

Magnolol—500, 250, 125, 62.5, 31.25, 15.63, 7.81, 3.91, 1.93, 0.98, 0.49 and 0

Sapienic acid: 125, 62.5, 31.25, 15.63, 7.81, 3.91, 1.95 and 0

D-Sphingosine: 20, 10, 5, 2.5, 1.25, 0.63, 0.31 and 0

D-erythro-Dihydrosphingosine: 40, 20, 10, 5, 2.5, 1.25, 0.63 and 0

Phytosphingosine: 40, 20, 10, 5, 2.5, 1.25, 0.63 and 0

Palmitoleic acid: 125, 62.5, 31.25, 15.63, 7.81, 3.91, 1.95 and 0

Pertaining to P. Acnes:

Honokiol—500, 250, 125, 62.5, 31.25, 15.63, 7.81, 3.91, 1.93, 0.98, 0.49 and 0

Magnolol—500, 250, 125, 62.5, 31.25, 15.63, 7.81, 3.91, 1.93, 0.98, 0.49 and 0

Sapienic acid—125, 62.5, 31.25, 15.63, 7.81, 3.91, 1.95 and 0

D-Sphingosine—20, 10, 5, 2.5, 1.25, 0.63, 0.31 and 0

D-erythro-Dihydrosphingosine—40, 20, 10, 5, 2.5, 1.25, 0.63 and 0

Phytosphingosine—40, 20, 10, 5, 2.5, 1.25, 0.63 and 0

Palmitoleic acid—125, 62.5, 31.25, 15.63, 7.81, 3.91, 1.95 and 0

Results:

It was observed that there was synergistic effect of honokiol and magnolol with AMLs against M. furfur (Table 1 and 2), S. aureus (Table 3 and 4) and P. acnes (Table 5 and Table 6).

TABLE 1
Effect of honokiol and AMLs against M. furfur

| | The range of concentration of | | The range of ratio |
|---|---|---|---|
| | the ingredient (ppm) where the | | of honokiol/AML |
| | ΣFIC was less than 0.9 | | (w/w) where ΣFIC |
| AML | honokiol | AML | was less than 0.9 |
| sapienic acid | 625 to 1250 | 625 to 2500 | 0.25 to 1 |
| sphingosine | 312.5 to 1250 | 31.25 to 250 | 1.25 to 40 |
| dihydrosphingosine | 625 to 1250 | 31.25 to 125 | 5 to 40 |
| phytosphingosine | 625 to 1250 | 15.63 to 62.5 | 10 to 80 |
| palmitoleic acid | 312.5 to 1250 | 625 to 5000 | 0.06 to 2 |

The data indicates a significantly wide range of concentration of honokiol and the concerned antimicrobial lipid that is at the disposal of formulation scientists which they could use to their advantage. The fact that ΣFIC was less than 0.9 across the broad ranges of honokiol when used in combination with any of the five AMLs, implies that is possible to formulate highly efficacious antimicrobial compositions comprising the two active ingredients after giving due consideration to the fact that the experiments were conducted under test conditions and the amount of the ingredients may not match or reflect the practical amounts under in-use conditions, i.e., compositions like a shampoo or a skin care cream.

The observations and inference drawn from the data in Table 1, apply mutatis mutandis to the data tabulated in Tables 2 to 6.

TABLE 2
Effect of magnolol and AMLs against M. furfur

| | The range of | | The range of |
|---|---|---|---|
| | concentration of the | | ratio of magnolol/ |
| | ingredient (ppm) where | | AML (w/w) |
| | the ΣFIC was less than 0.9 | | where ΣFIC |
| AML | magnolol | AML | was less than 0.9 |
| sapienic acid | 312.5 to 1250 | 625 to 5000 | 0.06 to 2 |
| sphingosine | 625 to 1250 | 31.25 to 125 | 5 to 40 |
| dihydrosphingosine | 625 to 1250 | 15.63 to 125 | 5 to 80 |
| phytosphingosine | 625 to 1250 | 3.91 to 62.5 | 10 to 320 |
| palmitoleic acid | 156.25 to 1250 | 625 to 5000 | 0.03 to 2 |

TABLE 3
Effect of honokiol and AMLs against S. aureus

| | The range of concentration of | | The range of ratio |
|---|---|---|---|
| | the ingredient (ppm) where | | of honokiol/AML |
| | the ΣFIC was less than 0.9 | | (w/w) where ΣFIC |
| AML | honokiol | AML | was less than 0.9 |
| sapienic acid | 0.49 to 7.81 | 3.91 to 62.5 | 0.008 to 2 |
| sphingosine | 0.98 to 7.81 | 0.31 to 1.25 | 0.8 to 25 |
| dihydrosphingosine | 1.95 to 7.81 | 0.63 to 2.50 | 0.8 to 12.5 |
| phytosphingosine | 1.95 to 7.81 | 0.63 to 5.00 | 0.4 to 12.5 |
| palmitoleic acid | 0.49 to 7.81 | 3.91 to 62.5 | 0.008 to 2 |

TABLE 4
Effect of magnolol and AMLs against S. aureus

| | The range of concentration | | |
|---|---|---|---|
| | of the ingredient (ppm) | | The range of ratio |
| | where the ΣFIC | | of magnolol/AML |
| | was less than 0.9 | | (w/w) where ΣFIC |
| AML | magnolol | AML | was less than 0.9 |
| sapienic acid | 0.49 to 7.81 | 1.95 to 31.25 | 0.01 to 4 |
| sphingosine | 0.98 to 7.81 | 0.31 to 0.63 | 1.5 to 25 |
| dihydrosphingosine | 0.49 to 7.81 | 0.63 to 1.25 | 0.4 to 12.5 |
| phytosphingosine | 0.49 to 7.81 | 0.63 to 2.50 | 0.2 to 12.5 |
| palmitoleic acid | 0.49 to 7.81 | 3.91 to 52.5 | 0.008 to 2 |

TABLE 5
Effect of honokiol and AMLs against P. acnes

| | The range of concentration of | | The range of ratio |
|---|---|---|---|
| | the ingredient (ppm) where | | of honokiol/AML |
| | the ΣFIC was less than 0.9 | | (w/w) where ΣFIC |
| AML | honokiol | AML | was less than 0.9 |
| sapienic acid | 0.98 to 1.95 | 1.95 to 3.91 | 0.25 to 1 |
| sphingosine | 0.98 to 3.91 | 0.31 to 1.25 | 0.7 to 13 |
| dihydrosphingosine | 0.49 to 3.91 | 0.63 to 2.50 | 0.2 to 6.5 |
| phytosphingosine | 0.98 to 3.91 | 1.25 to 5.00 | 0.2 to 3.2 |
| palmitoleic acid | 0.49 to 1.95 | 1.95 to 7.81 | 0.06 to 1 |

TABLE 6
Effect of magnolol and AMLs against P. acnes

| | The range of concentration of | | The range of ratio |
|---|---|---|---|
| | the ingredient (ppm) where | | of magnolol/AML |
| | the ΣFIC was less than 0.9 | | (w/w) where ΣFIC |
| AML | magnolol | AML | was less than 0.9 |
| sapienic acid | 0.98 to 1.95 | 1.95 to 3.91 | 0.25 to 1 |
| sphingosine | 1.95 to 3.91 | 0.31 to 1.25 | 1.5 to 13 |
| dihydrosphingosine | 1.95 to 3.91 | 0.63 to 2.50 | 0.7 to 6.5 |
| phytosphingosine | 1.95 to 7.81 | 0.63 to 5.00 | 0.4 to 12.5 |
| palmitoleic acid | 0.98 to 7.81 | 1.95 to 7.81 | 0.1 to 4 |

Therefore, in summary, the observations tabulated in Tables 1 to 6 clearly indicate that an antimicrobial lipid found in the sebum or stratum corneum of human beings (other than saturated C_{8 to 18} fatty acids) interacts synergistically with a biphenol obtainable from Magnolia spp. Their interaction was found to be synergistic across broad ranges of concentration and the synergistic interaction was evident from the fact that the ΣFIC was less than 0.9.

All the experiments disclosed hereinabove were conducted under in vitro conditions to ascertain whether the combination of an antimicrobial lipid found in the sebum or stratum corneum of human beings, other than saturated c8-c18 fatty acids and a biphenol obtainable from Magnolia spp was synergistic, additive or antagonistic vis-a-vis their individual activity against the concerned microbe. As far as the experiments were concerned, the concentrations of the ingredients were chosen to fall within the allowable limits permitted by the concerned tests and in which it was possible to record the technical effects. Therefore, the concentrations at which the tests were conducted might not appear to fall within the range in which such ingredients are generally used in cosmetic compositions (usually in wt %).

The compositions of the invention may be formulated as an emulsion or a gel with other usual ingredients which may affect the concentration of the desired actives in the oil phase and in the water phase. Such compositions might also have a different set of physical and hydrodynamic properties like partition coefficients, diffusional rates, convective transport rates and rheological properties. Therefore, it is expected that the concentrations to be used when formulated as a composition could be different from that at the cellular levels at which the experiments were carried out and usually the in-use concentrations are orders of magnitude higher.

1. A topical composition comprising an antimicrobial lipid found in the sebum or stratum corneum of human beings, other than saturated C₈₋₁₈ fatty acids, wherein said composition further comprises a biphenol obtainable from Magnolia spp.

2. The topical composition according to claim 1, wherein the amount of said lipid is 0.01 to 10 wt %.

3. The topical composition accroding to claim 1, wherein the amount of said biphenol obtainable from Magnolla spp. is 0.01 to 10 wt %.

4. The topical composition according to claim 1, wherein said lipid is at least one of sapienic acid, sphigosine, dihydrosphingosine, phytosphingosine, 6-hydroxy phytosphingosine or palmitoleic acid.

5. The topical composition according to claim 1, comprising water- or hydro-alcoholic extract of the bark of Magnolia spp., which in turn comprises said biphenol.

6. The topical composition according to claim 1, wherein said biphenol is at least one of honokiol or magnolol.

7. The topical composition according to claim 6, wherein said composition comprises honokiol and magnolol.

8. The topical composition according to claim 1, wherein said composition comprises 0.01 to 10 wt % of said honokiol or 0.01 to 10 wt % of said magnolol, provided that when said composition comprises both honokiol and magnolol, then their combined amount is 0.01 to 10 wt % of said composition.

9. A method comprising the step of topically applying a topical composition of claim 1 to the skin as a topical antimicrobial composition.

10. The method according to claim 9, wherein said topical composition is an antiacne composition effective at least against P. acnes.

11. The method according to claim 9, wherein said topical composition is an antidandruff composition effective against at least some Malassezia spp.

12. The method according to claim 11, wherein said topical composition is rinse-off or a leave-on composition effective at least against S. aureus.

13. The method according to claim 9, wherein said method is non-therapeutic.

14. The method according to claim 9, wherein said method is for a cosmetic purpose.

15. A method of providing topical antimicrobial benefit comprising a step of applying a safe and effective amount of a topical antimicrobial composition of claim 1.

16. The topical composition according to claim 1, wherein the amount of said antimicrobial lipid to said biphenol is 0.003 to 125.

17. The topical composition according to claim 11, wherein the composition further comprises a zinc compound, conazole fungicide or mixture thereof.

18. The topical composition according to claim 1, wherein the composition has a pH equal to or higher than 4.0.
